(19) United States
(12) Patent Application Publication
(10) Pub. No.: US 2007/0197903 A1
(43) Pub. Date: Aug. 23, 2007

(54) System and method for improved detection and assessment of changes in lung-tissue structure

(76) Inventors: John P. Mugler III, Charlottesville, VA (US); James R. Brookeman, Charlottesville, VA (US); Jaime F. Mata, Richmond, VA (US); Talissa A. Altes, Philadelphia, PA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/592,186

(22) PCT Filed: Mar. 9, 2005
(86) PCT No.: PCT/US05/08058

§ 371 Date: Sep. 8, 2006

Related U.S. Application Data
(60) Provisional application No. 60/551,877, filed on Mar. 10, 2004.

(51) Int. Cl. A61B 5/05

(52) U.S. Cl. 600/420

(57) ABSTRACT

A method and system is described for measuring the apparent diffusion coefficient of Xe129 in the lung as a means to detect and assess changes in lung-tissue structure such as those that occur in certain pulmonary diseases. The main steps of this process include: polarizing the Xe129 gas; introducing said gas into the lung; acquiring sets of Xe 129 M R signals with various diffusion sensitizations; calculating Xe 129 ADC values; and evaluating said ADC values by comparison of the values in a region of interest to those in different regions of the lung or to normative values.

### CROSS-REFERENCE TO RELATED APPLICATIONS

This application claims priority from U.S. Provisional Application No. 60/551,877, filed on Mar. 10, 2004, entitled “System and Method for Improved Detection and Assessment of Changes in Lung Tissue Structure,” the disclosure of which is hereby incorporated by reference in its entirety.

The present application is also related to PCT International Application No. PCT/US05/xxxxx, filed Mar. 9, 2005 which claimed priority to Provisional Application No. 60/551,884), entitled “Exchange-weighted Xenon-129 Nuclear Magnetic Resonance System and Related Method,” of which are assigned to the present assignee and are hereby incorporated by reference herein in their entirety. The present invention may be implemented with the technology discussed throughout aforementioned International Application entitled “Exchange-weighted Xenon-129 Nuclear Magnetic Resonance System and Related Method.”

### FIELD OF THE INVENTION

The present invention relates generally to nuclear magnetic resonance imaging, and more particularly to the application of diffusion magnetic resonance imaging methods to hyperpolarized Xenon-129 (“Xe129”).

### BACKGROUND OF INVENTION

Chronic Obstructive Pulmonary Disease (“COPD”) is a slowly progressive disease of the airways that is characterized by a gradual loss of lung function. COPD, which includes chronic bronchitis and emphysema, is the most common chronic lung disease and the fourth leading cause of death in the United States. As treatments for COPD continue to advance, a more rigorous clinical assessment of disease severity and distribution has become relevant. Unfortunately, current techniques for the evaluation of COPD possess notable limitations: pulmonary function tests are insensitive to early changes in the lung; radiographic examination of the lungs is of limited use since pulmonary lobules cannot be visualized with conventional radiography; thin-section computed topography has limited sensitivity in the detection of early emphysematous changes. See Takasugi J E, Godwin J D, “Radiology of Chronic Obstructive Pulmonary Disease,” Radiol Clin North Am 1998; 36:29-55; Gurney J W, “Pathophysiology of Obstructive Airways Disease,” Radiol Clin North Am 1998; 36:15-27, of which are hereby incorporated by reference herein in their entirety.

The application of hyperpolarized noble gasses in magnetic resonance imaging was a significant improvement over previous methods. See, e.g., Pines et al., U.S. Pat. No. 6,426,058 B1, entitled “Enhancing of NMR and MRI in the Presence of Hyperpolarized Noble Gases” and Albert et al., U.S. Pat No. 6,241,966 B1, “Magnetic Resonance Imaging Using Hyperpolarized Noble Gases,” of which are herein incorporated by reference in their entirety. The application of hyperpolarized Helium-3 (“He3”) magnetic resonance imaging is capable of producing high-spatial-resolution magnetic resonance images of the lung air spaces after the inhalation of the gas. See Middleton H, Black R D, Saam B, et al., “M R Imaging with Hyperpolarized He-3 Gas,” Magn Reson Med 1995; 33:271-275; Black R D, Middleton H L, Cates G D, et al., “In vivo He-3 MR Images of Guinea Pig Lungs,” Radiology 1996; 199:867-870; Kauczor H U, Hofmann D, Kreitner K F, et al., “Normal and Abnormal Pulmonary Ventilation: Visualization at Hyperpolarized He-3 MR Imaging,” Radiology 1996; 201:564-568; and MacFall J R, Charles H C, Black R D, et al., “Human Lung Air Spaces: Potential for MR Imaging with Hyperpolarized He-3,” Radiology 1996; 200:553-558, of which are hereby incorporated by reference herein in their entirety.

Hyperpolarized He3 ventilation magnetic resonance imaging has shown moderate success for differentiating healthy lungs from those with disease, but, this method does not provide information about the integrity of the lung microstructure in the ventilated regions (See Kauczor H U, Ebert M, Kreitner K F, et al., “Imaging of the Lungs using ³He MRI: Preliminary Clinical Experience in 18 Patients with and without Lung Disease,” J Magn Reson Imaging 1997; 7:538-543; de Lange E E, Mugler J P III, Brookeman J R, et al., “Lung Air Spaces: MR Imaging Evaluation with Hyperpolarized ³He Gas,” Radiology 1999; 210:851-857, of which are hereby incorporated by reference herein in their entirety), asthma (See Altes T A, Powers P L, Knight-Scott J, et al., “Hyperpolarized ³He MR Lung Ventilation Imaging in Asthmatics: Preliminary Findings,” J Magn Reson Imaging 2001; 13:378-384, of which is hereby incorporated by reference herein in it entirety), and cystic fibrosis (See Donnelly L F, MacFall J R, McAdams H P, et al., “Cystic Fibrosis: Combined Hyperpolarized ³He-enhanced and Conventional Proton MR Imaging in the Lung—Preliminary Observations,” Radiology 1999; 212:885-889, of which is hereby incorporated by reference herein in it entirety).

In a recently developed technique, He3 diffusion magnetic resonance imaging allows the lung microstructure to be probed. (Concerning diffusion in conventional proton magnetic resonance imaging, see Moseley M E, Cohen Y, Mintorovitch J, et al., Early Detection of Regional Cerebral Ischemia in Cats: Comparison of Diffusion- and T2-weighted MRI and Spectroscopy,” Magn Reson Med 1990; 14:330-346, of which is hereby incorporated by reference herein in their entirety.) It has been demonstrated that regional quantification of the lung microstructure is possible by combining the high diffusivity of He3 with established MR methods for measuring diffusion, thereby providing quantitative spatial maps of the apparent diffusion coefficient (ADC) of He3 in the lung. See Mugler J P III, Brookeman J R, Kight-Scott J, et al., “Regional Measurement of the ³He Diffusion Coefficient in the Human Lung,” In: Proc Intl Soc Magn Reson Med, 6th Meeting, 1998; 1906; Chen X J, Moller H E, Chawla M S, et al., “Spatially Resolved Measurements of Hyperpolarized Gas Properties in the Lung in Vivo. Part I: Diffusion Coefficient,” Magn Reson Med 1999; 42:721-728; and Saam B T, Yablonskiy D A, Kodibagkar V D, et al., “MR Imaging of Diffusion of ³He Gas in Healthy and Diseased Lungs,” Magn Reson Med 2000; 44:174-179, of which are hereby incorporated by reference herein in their entirety. He3 has a high self-diffusion coefficient. When He3 is confined to spaces, such as the airway spaces in the lung, its motion is restricted, which results in smaller displacement and a decrease in the ADC as measured with magnetic resonance imaging. The ADC provides a measure of the distance over which a He3 atom can travel during a selected time period. This distance depends on the microscopic characteristics of the lung structure. As a result, changes in the lung microstructure, as can occur in lung disease, give rise to changes in the ADC value of He3. By comparing ADC maps obtained from healthy subjects to those obtained from patients with lung disease, microstructural changes can be studied. See Chen X J, Hedlund L W, Moller H E, et al., “Detection of Emphysema in Rat Lungs by Using Magnetic Resonance Measurements of ³He Diffusion,” Proc Natl Acad Sci USA 2000; 97:11478-11481; Salerno M, de Lange E E, Altes T A, et al., Emphysema: Hyperpolarized Helium 3 Diffusion MR Imaging of the Lungs Compared with Spirometric Indexes—Initial Experience,” Radiology 2002; 222:252-260; and Yablonskiy D A, Sukstanskii A L, Leawoods J C, et al., “Quantitative in Vivo Assessment of Lung MicroStructure at the Alveolar Level with Hyperpolarized ³He Diffusion MRI,” Proc Natl Acad Sci USA 2002; 99:3111-3116, of which are hereby incorporated by reference herein in their entirety.

Unfortunately, due to the high diffusivity He3, diffusion magnetic resonance imaging is of limited usefulness. The ADC for He3 in the healthy human lung is approximately 0.2 cm²/s. Based on established relationships for predicting displacements due to diffusion, this value yields a root-mean-squared (“RMS”) displacement along one dimension of 350 μm during the diffusion-sensitization period (on the order of 3 milliseconds) typically used for bipolar-gradient-based diffusion techniques. Considering that the diameter of a health human alveolus is approximately 250 μm, it appears that He3 atoms typically visit several alveoli during the diffusion-sensitization period. Thus, it seems that disease effects would need to be significant at the level of the alveolar ducts, or perhaps even the pulmonary acini, to substantially affect the ADC of He3.

Accordingly, the present invention application of diffusion magnetic resonance imaging methods to hyperpolarized Xe129 provides the ability to better detect the early stages of lung-tissue destruction that occur. The ADC for Xe129 in the healthy human lung is approximately 0.04 cm²/s. See Mugler J P III, Mata J F, Wang H T J, et al., “The Apparent Diffusion Coefficient of Xe-129 in the Lung: Preliminary Human Results,” In: Proc Intl Soc Magn Reson Med, 12th Meeting, 2004, of which is hereby incorporated by reference herein in its entirety. This value yields a RMS displacement along one dimension of 150 μm during the diffusion-sensitization period typically used for bipolar-gradient-based diffusion techniques. Considering that the diameter of a healthy human alveolus is approximately 250 μm, it appears that Xe129 only visits one or two alveoli during the diffusion-sensitization period. Thus, diffusion imaging with Xe129 will likely yield increased sensitivity for the detection of certain pulmonary pathologies compared to established methods. This increased sensitivity, combined with the fact that Xe129 is a natural component of the atmosphere, whereas He3 is a rare isotope that comes primarily from the decay of tritium and is therefore relatively expensive and in very limited supply, suggests that diffusion imaging with Xe129 may develop into the method of choice for detecting and quantifying changes in lung-tissue structure.

### SUMMARY OF INVENTION

In summary, various embodiments of the invention comprise using magnetic resonance imaging of hyperpolarized Xe129 to permit the detection and quantitative assessment of changes in lung-tissue structure such as occur in certain pulmonary diseases, but not limited thereto. Compared to existing methods, the various embodiments of the present invention provide the potential to detect lung-tissue destruction at an earlier stage, which may be useful to provide earlier diagnosis and quantitative assessment of lung-tissue injury secondary to diseases such as emphysema, permitting improved patient care, and which may be valuable as a means to aid in the formulation and quantitative evaluation of new respiratory drugs.

The various embodiments of the present invention, diffusion imaging with Xe129, represent a novel approach for assessing changes in lung-tissue structure. Various embodiments of the invention measure the diffusion of hyperpolarized Xe129 to achieve increased sensitivity for the detection and monitoring of certain pulmonary pathologies. Diffusion measurements with Xe129 will likely yield increased sensitivity for the detection of certain pulmonary pathologies compared to established methods because the lengths that can be probed with Xe129 are: (i) smaller than those that can be probed with established methods that are based on He3 and (ii) similar to the physical dimensions of healthy alveoli. This increased sensitivity is combined with the fact that Xe129 is a natural component of the atmosphere, whereas He3 is a rare isotope on Earth, comes primarily from the decay of tritium and is therefore relatively expensive and in very limited design. As such, the various embodiments of diffusion imaging with Xe129 may develop into the method of choice for detecting and quantifying changes in lung-tissue structure, among other things.

An aspect of an embodiment of the present invention includes the following steps: polarizing the Xe129 gas; introducing said gas into the lung; acquiring sets of Xe129 MR signals with various diffusion sensitizations; calculating Xe129 ADC values; and evaluating said ADC values by comparison of the values in a region of interest to those in different regions of the lung or to normative values.

Various embodiments of the present invention feature, but are not limited thereto, a method and apparatus for detecting and assessing changes in lung-tissue structure by measuring the ADC of Xe129 in a lung using a magnetic resonance imaging system. An aspect of an embodiment of the method for detecting or assessing changes in lung-tissue in a lung comprises: a) generating hyperpolarized Xe129 gas; b) introducing the gas into the lung after the lung is positioned within an appropriate radio-frequency coil that is within a magnetic resonance imaging (MRI) apparatus; c) acquiring at least two magnetic resonance signals from Xe129 nuclei within the lung wherein the signals are “diffusion sensitized” such that the value of a property of the signals varies between the signals and reflects, among other possible effects, the degree to which diffusion has affected the signals; d) calculating apparent diffusion coefficient values from the diffusion-sensitized magnetic resonance signals; and e) evaluating apparent diffusion coefficient values.

An aspect of an embodiment of the MRI apparatus for detecting or assessing changes in lung-tissue using hyperpolarized Xe129 gas comprises: a) a radio frequency coil, wherein the gas is introduced into the lung and the lung is positioned within the radio-frequency coil; b) an MR images acquisition means, wherein the MR images acquisition means acquiring at least two magnetic resonance signals from Xe129 nuclei within the lung wherein the signals are “diffusion sensitized” such that the value of a property of the signals varies between the signals and reflects, among other possible effects, the degree to which diffusion has affected the signals; c) calculating means, wherein the calculating means for calculating apparent diffusion coefficient values from the diffusion-sensitized resonance signals; and d) an evaluating means, wherein the evaluating means for evaluating coefficient values.

These and other objects, along with advantages and features of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 illustrates a simplified exemplary embodiment of a MRI apparatus for practicing the present invention. The present invention method can be applied to various commercially available MRI apparatuses.

FIG. 2 shows coronal (A) hyperpolarized Xe129 and (B) hyperpolarized He3 ADC maps from the lung of a New Zealand rabbit with induced emphysema in the right lung.

FIG. 3 shows a diagram identifying the steps of an exemplary embodiment of the method of the present invention.

### DETAILED DESCRIPTION OF THE INVENTION

One type of molecular motion which can affect the MRI signal is molecular 20 diffusion, which consists of the random motion. Xe129 has a moderately high self-diffusion coefficient of 0.14 cm²/s. See Chen X J, Moller H E, Chawla M S, et al., “Spatially Resolved Measurements of Hyperpolarized Gas Properties in the Lung in Vivo. Part I: Diffusion Coefficient,” Magn Reson Med 1999; 42:721-728, of which is hereby incorporated by reference herein in its entirety. However, when Xe129 is confined to spaces, such as the airway structure of the lung, its motion is restricted. Since Xe129 gas in the lung is in the restricted regime, the term apparent diffusion coefficient is used for the parameter that is measured by MRI diffusion experiments. The ADC for Xe129 in the healthy human lung is approximately 0.04 cm²/s. See Mugler J P III, Mata J F, Wang H T J, et al., “The Apparent Diffusion Coefficient of Xe-129 in the Lung: Preliminary Human Results,” In: Proc Intl Soc Magn Reson Med, 12th Meeting, 2004, of which is hereby incorporated by reference herein in its entirety. Based on the established relationships for predicting displacements due to diffusion, this value yields a RMS displacement along one dimension of 150 μm during the diffusion-sensitization period typically used for bipolar-gradient-based diffusion techniques. The diameter of a healthy human alveolus is approximately 250 μm. Thus, it appears that Xe129 atoms typically visit only one or two alveoli during a diffusion sensitization period of a few milliseconds. Therefore, the use of Xe129 may detect earlier lung tissue destruction compared to established methods that are based on He3, which typically visits several alveoli during the diffusion-sensitization period.

MR imaging is a non-invasive technique available to measure diffusion. Diffusion-weighted MR imaging exploits the random motion of the molecules, which causes a phase dispersion of the spins with a resultant signal loss. Using MRI to assess diffusion is attractive because it allows accurate control of the diffusion direction and the time during which diffusion takes place. Also, the mean diffusion path length or displacement of the diffusing atoms or molecules can be determined.

Numerous MR methods have been developed to measure diffusion-induced signal changes. See, e.g., Lang et al., U.S. Pat. No. 5,671,741, entitled “Magnetic Resonance Imaging Technique for Tissue Characterization,” of which is hereby incorporated by reference herein in its entirety.

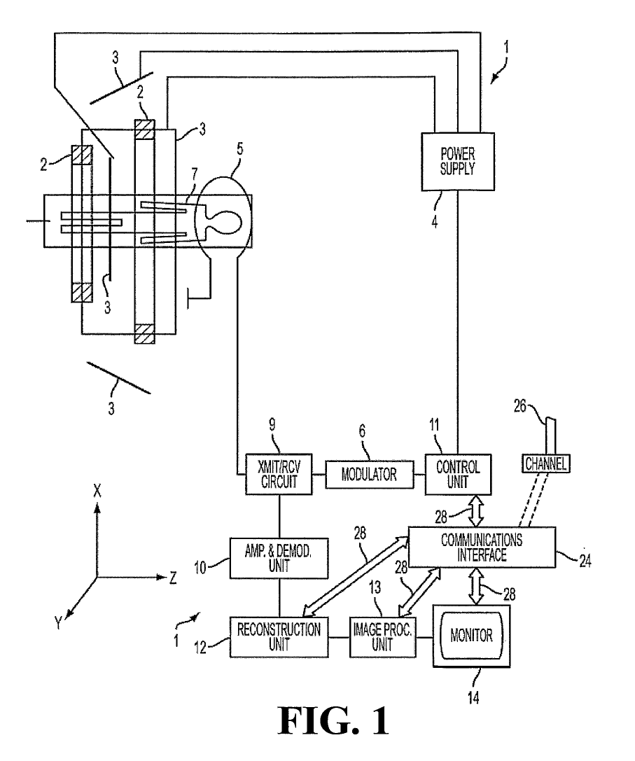

FIG. 1 illustrates a simplified schematic of a MR apparatus 1 or scanner for practicing an embodiment of the present invention. The MR apparatus 1 includes a main magnet system 2 for generating a steady magnetic field in an examination zone(s) of the MR apparatus. The z-direction of the coordinate system illustrated corresponds to the direction of the steady magnetic field generated by the magnet system 2.

The MR system or scanner also includes a gradient magnet system 3 for generating temporary magnetic fields Gₓ, G_{y} and G_{z} directed in the z-direction but having gradients in the x, y or z directions, respectively. With this magnetic gradient system, magnetic-field gradients can also be generated that do not have directions coinciding with the main directions of the above coordinate system, but that can be inclined thereto, as is known in the art. Accordingly, the present invention is not limited to directions fixed with respect to the MR system.

Also, while traditional commercial methods provide linear gradients in the x, y, or z directions it is also possible not to utilize all three of these linear gradients. For example, rather than using a linear z gradient, one skilled in the art can use a z-squared dependence or some other spatial dependence to provide desired results.

The magnet systems 2 and 3 enclose an examination zone(s) which is large enough to accommodate a part of an object 7 to be examined, for example a part of a human patient. A power supply means 4 feed the gradient magnet system 3.

The MR system also includes an RF transmitter system including RF transmitter coil 5, which generates RF pulses in the examination zone and is connected via transmitter/receiver circuit 9 to a RF source and modulator 6.

The RF transmitter coil 5 is arranged around the part of body 7 in the examination zone. The MR apparatus also comprises an RF receiver system including an RF receiver coil that is connected via transmitter/receiver circuit 9 to signal amplification and demodulation unit 10. The receiver coil and the RF transmitter coil 5 may be one and the same coil.

A gas supply (and/or gas regulator), not shown, provides hyperpolarized Xe129 gas to the examination zone or region of the object/subject (body, cavity, or the like). The gas supply may be an attachable supply line to the object/subject or may be a portable gas supply such as a container, bolus delivery device, or dose bag. As would be appreciated by one skilled in the art, there are wide variety of methods and systems adapted for supplying hyperpolarized gas to the object or subject (or region and examination zone). For illustrative examples of magnetic resonance imaging that may or may not use hyperpolarized gases include the following patents and patent applications and are hereby incorporated by reference herein in their entirety: 1) commonly assigned U.S. Pat. No. 5,245,282, filed Jun. 28, 1991, entitled “Three-dimensional Magnetic Resonance Imaging,” 2) co-assigned U.S. Pat. No. 6,630,126 B2, filed Mar. 12, 2001, entitled “Diagnostic Procedures Using Direct Injection of Gaseous Hyperpolarized 129Xe and Associated Systems and Products,” and its corresponding International Patent Application Serial No. PCT/US01/07812, filed Mar. 12, 2001 (Publication No.: WO/01/67955 A2), 3) co-assigned U.S. Pat. No. 6,775,568 B2, filed Apr. 12, 2001, entitled “Exchange-Based NMR Imaging and Spectroscopy of Hyperpolarized Xenon-129,” 4) pending and commonly assigned U.S. patent application Ser. No. 10/451,124, filed Jun. 19, 2003, entitled “Method and Apparatus for Spin-echo-train MR Imaging Using Prescribed Signal Evolutions” and corresponding International Patent Application Serial No. PCT/US01/50551, filed Dec. 21, 2001, entitled “Method and Apparatus for Spin-echo-train MR Imaging Using Prescribed Signal Evolutions,” 5) pending and commonly assigned U.S. patent application Ser. No. 10/474,571, filed Oct. 14, 2003, entitled “Optimized High Speed Magnetic Resonance Imaging Method and System Using Hyperpolarized Noble Gases” and corresponding International Patent Application Serial No. PCT/US02/11746, filed Apr. 12, 2002, entitled “Optimized High Speed Magnetic Resonance Imaging Method and System Using Hyperpolarized Noble Gases”, and 6) pending and commonly assigned International Patent Application Serial No. PCT/US03/151136, filed May 14, 2003, entitled “Method and System for Rapid Magnetic Resonance Imaging of Gases with Reduced Diffusion-induced Signal Loss.”

Some illustrative examples of magnetic resonance imaging that may or may not use hyperpolarized gases are provided in the following patent applications and patents and are hereby incorporated by reference herein in their entirety: U.S. Pat. No. 5,545,396 to Albert et al., entitled “Magnetic Resonance Imaging Using Hyperpolarized Noble Gases;” U.S. Pat. No. 5,785,953 to Albert et al., entitled “Magnetic Resonance Imaging Using Hyperpolarized Noble Gases;” and U.S. Pat. No. 5,789,921 to Albert et al., entitled “Magnetic Resonance Imaging Using Hyperpolarized Noble Gases.” Some aspects of some embodiments of the present invention may be implemented with the technology discussed in U.S. Pat. No. 6,491,895 B2 to Driehuys et al., entitled “Method for Imaging Pulmonary and Cardiac Vasculature and Evaluating Blood Flow Using Dissolved Polarized XE129,” and U.S. Pat. No. 5,492,123 to Edelman, entitled “Diffusion Weighted Magnetic Resonance Imaging.”

The MR system or scanner also includes an amplification and demodulation unit or system 10, which, after excitation of nuclear spins in a part of the body placed within the examination space by RF pulses, after encoding by the magnetic-field gradients and after reception of the resulting MR signals by the receiver coil, derives sampled phases and amplitudes from the received MR signals. An image reconstruction unit or system 12 processes the received MR imaging signals to, inter alia, reconstruct an image by methods well-known in the art, such as by Fourier transformation. It should be appreciated by one skilled in the art that various reconstruction methods may be employed besides the Fourier Transform (FT) depending on factors such as the type of signal being analyzed, the available processing capability, etc. For example, but not limited thereto, the present invention may employ Short-Time FT (STFT), Discrete Cosine Transforms (DCT), or wavelet transforms (WT). By means of an image processing unit or system 13, the reconstructed image is displayed, for example, on monitor 14. Further, the image reconstruction unit or system can optionally process MR navigator signals to determine the displacement of a portion of the patient.

The MR system also includes a control unit or system 11 that generates signals for controlling the RF transmitter and receiver systems by means of a modulator 6, the gradient magnetic field system by means of the power supply means 4, an image reconstruction unit or system 12 and an image processing unit or system 13. In an exemplary embodiment, the control unit or system 11 (and other control elements in the MR system) are implemented with programmable elements, such as one or more programmable signal processors or microprocessors, communicating over busses with supporting RAM, ROM, EPROM, EEPROM, analog signal interfaces, control interfaces, interface to computer-readable media and so forth. These programmable elements are commanded by software or firmware modules loaded into RAM, EPROM, EEPROM or ROM, written according to well-known methods to perform the real-time processing required herein, and loaded from computer-readable media (or computer useable medium), such as magnetic disks or tapes, or optical disks, or network interconnections, removable storage drives, flash memory, or so forth. The present invention may be implemented using hardware, software or a combination thereof and may be implemented in one or more computer systems or processing systems, such as personal digit assistants (PDAs), for various applications, e.g., remote care and portable care practices.

In an embodiment, the control unit that directs a MR system for practicing the present invention can be implemented with dedicated electronic components in fixed circuit arrangements. In this case, these dedicated components are arranged to carry out the method described above. For example, the invention is implemented primarily in hardware using, for example, hardware components such as application specific integrated circuits (ASICs). Implementation of the hardware state machine to perform the functions described herein will be apparent to persons skilled in the relevant art(s).

In particular, the control unit commanded by its loaded software causes the generation of MR signals by controlling the application of MR pulse sequences, which comprise RF-pulses, time delays and temporary magnetic-field gradient pulses. These pulse sequences are generated according to the methods of the present invention as subsequently described, and generally include 2D and 3D imaging pulse sequences and optionally navigator pulse sequences for determining the displacement of the patient or material.

Furthermore, according to alternate embodiments of the present invention, the MR system also optionally includes various other units (not illustrated) from which the state of motion of the part of the patient being imaged can be measured. These can include sensors directly indicating the instantaneous state of motion of the part of the patient being imaged, such as a chest belt for directly indicating chest displacement during respiration, or MR-active micro-coils whose position can be tracked, or optical means, or ultrasound means, or so forth. These units can also include sensors indirectly indicating the instantaneous state of motion of the part of the patient being imaged. For example, electrocardiogram and peripheral pulse sensors measure the temporal progress of the cardiac cycle, and permit inference of the actual state of motion of the heart from knowledge of cardiac displacements associated with each phase of the cardiac cycle. When these sensors are present to measure the state of motion, the control unit need not generate navigator pulse sequences.

Moreover, the control unit or system 11 may also include a communications interface 24. The communications interface 24 allows software and data to be transferred between and among, via communication path (i.e., channel) 28 the control unit or system 11, reconstruction unit or system 12, image processing unit or system 13, and monitor 14 and external devices. Examples of the communications interface 24 may include a modem, a network interface (such as an Ethernet card), a communications port, a PCMCIA slot and card, etc. Software and data transferred via communications interface 24 are in the form of signals that may be electronic, electromagnetic, optical or other signals capable of being received by communications interface 24. The signals are provided to communications interface 24 via the communications path (i.e., channel) 26. The channel 26 carries signals and may be implemented using wire or cable, fiber optics, a phone line, a cellular phone link, a RF link, IR link, Bluetooth, and other communications channels.

Some embodiments of the present invention may be implemented as software/firmware/hardware with various MR systems, and methods, as one skilled in the art would appreciate. Other exemplary systems and methods, but not limited thereto, are disclosed in the following U.S. Patents, of which are hereby incorporated by reference in their entirety herein: U.S. Pat. No. 6,281,681 B1 to Cline et al., entitled “Magnetic Resonance Imaging with Interleaved Fibonacci Spiral,” U.S. Pat. No. 6,230,039 B1 to Stuber et. al., entitled “Magnetic Resonance Imaging Method and System with Adaptively Selected Flip Angles,” U.S. Pat. No. 5,749,834 to Hushek, entitled “Intersecting Multislice MRI Data Acquisition Method,” U.S. Pat. No. 5,656,776 to Kanazawa, entitled “Magnetic Resonance Imaging Apparatus,” U.S. Pat. No. 5,604,435 to Foo et al., entitled “Spiral Scanning Method for Monitoring Physiological Changes,” and U.S. Pat. No. 5,485,086 to Meyer et al, entitled “Continuous Fluoroscopic MRI Using Spiral K-space Scanning.”

The various forms of the present invention involve a method for detecting and quantifying pathological changes in the microstructure of the lung by measuring the ADC of Xe129 with magnetic resonance imaging. The method generally can be summarized as generally presented below.

Generating hyperpolarized Xe129, wherein we define the “hyperpolarized” state as a large (relative to the thermal equilibrium polarization for the polarizable gas in the static magnetic field used to acquire the MR images), non-equilibrium nuclear polarization. The Xe129 may be hyperpolarized for use according to the invention through any of various means known in the art, such as spin-exchange interactions with optical pumping. See Walker T G, Happer W., “Spin-exchange Optical Pumping of Noble Gas Nuclei”, Rev Mod Phys 1997; 69:629-642, of which is hereby incorporated by reference herein in its entirety. The volume and nuclear polarization of the hyperpolarized Xe129 gas are chosen based on the volume of the lung and on the desired spatial resolution, temporal resolution and signal-to-noise ratio of the MR signals to be generated.

Positioning the lung within an appropriate magnetic resonance system. The lung may be that of a human or an animal, and may be in vivo or excised.

Introducing the Xe129 into the lung (for example after the subject or excised lung is positioned with an appropriate radio-frequency coil that is within an MR scanner) using any available method that does not completely depolarize the gas. This includes but is not limited to the introduction into the lung by inhalation from a plastic bag, inhalation from a computer controlled gas mixing system, introduction by depressing a gas-filled syringe, or introduction by using a computer-controlled or manually-controlled ventilation device. It may be desirable to mix other gases with the Xe129 prior to or during the inhalation process as a means to fine-tune the diffusion characteristics of the gas mixture in the lung.

Acquiring MR signals from Xe129 nuclei within the lung wherein these signals are “diffusion sensitized” such that the value of a property of the signals varies between the signals and reflects, among other possible effects, the degree to which diffusion has affected the signals. In addition, the MR signals may be spatially-encoded using any appropriate imaging pulse sequence known in the art to generate images at one or more spatial locations, having any desired orientation relative to established anatomic landmarks, acquired for at least two diffusion-sensitization values and for at least one diffusion direction. This acquisition may use any MR technique suitable for gas-diffusion imaging, including, but not limited to, a gradient-echo pulse sequence that incorporates a bipolar gradient wave form just after the excitation radio-frequency pulse for diffusion sensitization. See Chen X J, Moller H E, Chawla M S, et al., “Spatially Resolved Measurements of Hyperpolarized Gas Properties in the Lung in Vivo. Part I: Diffusion Coefficient,” Magn Reson Med 1999; 42:721-728; Saam B T, Yablonskiy D A, Kodibagkar V D, et al., “MR Imaging of Diffusion of ³He Gas in Healthy and Diseased Lungs,” Magn Reson Med 2000; 44:174-179; Chen X J, Hedlund L W, Moller H E, et al., “Detection of Emphysema in Rat Lungs by Using Magnetic Resonance Measurements of ³He Diffusion,” Proc Natl Acad Sci USA 2000; 97:11478-11481; Salerno M, de Lange E E, Altes T A, et al., “Emphysema: Hyperpolarized Helium 3 Diffusion MR Imaging of the Lungs Compared with Spirometric Indexes—Initial Experience,” Radiology 2002; 222:252-260; and Yablonskiy D A, Sukstanskii A L, Leawoods J C, et al., Quantitative in Vivo Assessment of Lung Microstructure at the Alveolar Level with Hyperpolarized ³He Diffusion MRI,” Proc Natl Acad Sci USA 2002; 99:3111-311 6, of which are incorporated by reference herein in their entirety. The MR signals may be acquired during inhalation, during exhalation, during breath-hold, after rebreathing the gas or for some combination of these conditions. Sets of diffusion-sensitized signals, or images for the case that spatial-encoding is applied, may be acquired at several time points over the period for which measurable signal can be obtained from the Xe129 in the lung.

Calculating ADC values from the diffusion-sensitized signals, or spatial maps of ADC values for the case that spatial-encoding is applied and diffusion-sensitized images are generated, by using any appropriate mathematical processing, including, but not limited to, linear least squares fitting of the natural logarithm of the signal intensities versus degree of diffusion sensitization. If more than one diffusion-sensitization direction is used, ADC values or maps (as appropriate) are calculated for each direction. As appropriate, ADC values or maps may also be calculated that combine the information from multiple diffusion-sensitization directions.

Evaluating the ADC values in any given region of interest by using any appropriate quantitative or statistical comparisons. Such appropriate means include, but are not limited to, comparing the means and standard deviations. The ADC values in any give region(s) of interest in the lung are compared to those for other regions of interest in the lung, or to normative ADC values, as a quantitative metric of the state of the corresponding tissue.

For various applications such as monitoring the progression of disease or evaluating the efficacy of therapy, any of the steps discussed above (generating gas, introducing gas, acquiring diffusion-sensitized MR signals, calculating ADC values or maps, and evaluating ADC values or maps) would be repeated as desired to determine the variation in the ADC values over time.

A specific implementation of this methodology is useful to illustrate the nature of an embodiment of the present invention. For this purpose, the sets of lungs referenced to are those of anesthetized New Zealand rabbits. Rabbits were anesthetized with a mixture of xylazine and ketamine, and intubated with an endotracheal tube. The study protocol was approved by the Institutional Animal Care and Use Committee.

The experimental results described below were acquired using a 1.5 Tesla whole-body MR scanner. Diffusion imaging was performed by using a gradient-echo-based pulse sequence with a bipolar diffusion-sensitization gradient (Xe-129: degree of diffusion sensitization, b=0, 5 and 10 s/cm²; He-3: b=0, 1.6 and 4 s/cm²). For xenon, 3 image slices were acquired with 20-mm thickness; matrix 72×128; minimum in-plane resolution 2.7×3.1 mm²; flip angle 10°. Isotopically-enriched Xe129 (85%; Spectra Gases, Alpha, N.J.) was used. For helium, 6 slices were acquired with 10-mm thickness; matrix 64×128; in-plane resolution 2.2×2.2 mm²; flip angle 10°. The images were taken during breath-hold.

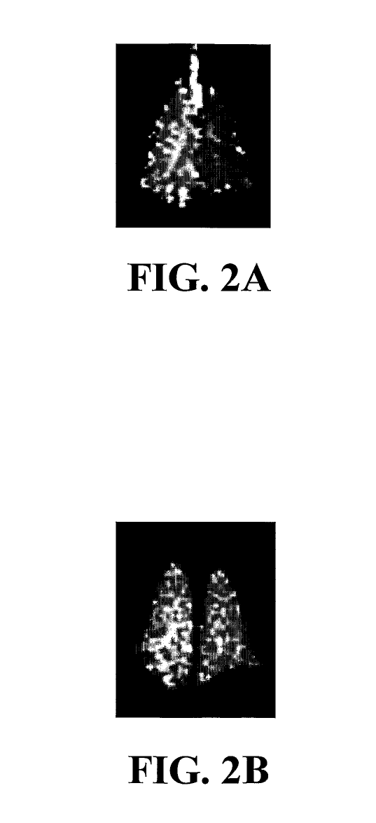

FIG. 2 shows an example of diffusion MR imaging with hyperpolarized Xe129 and provides preliminary evidence that Xe129 ADC measurements, as shown in FIG. 2(A), may yield an improvement in sensitivity to lung-tissue destruction compared to He3 ADC measurements, as shown in FIG. 2(B). The figure shows ADC maps from a New Zealand rabbit for which elastase was injected into the right lung over a four week period at 7-day intervals to induce emphysema. By week 8, the disease had progressed so that both Xe129 (FIG. 2(A)) and He3 (FIG. 2(B)) ADC values became elevated compared to baseline measurements. However, the mean Xe129 ADC value in the right lung was elevated by 42% whereas the corresponding He3 ADC value had increased by only 27%.

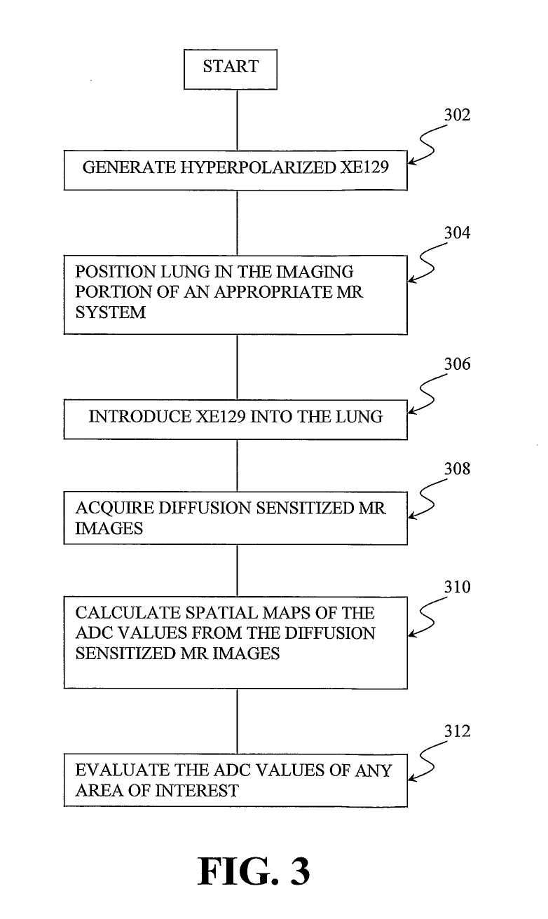

FIG. 3 is a diagram describing some steps set forth as the exemplary embodiment of the present invention. Upon starting the method, at step 302 the Xe129 is hyperpolarized and prepared for introduction into the lung. At step 304, the lung is placed into the imaging portion of a MR system in preparation for MR imaging. At step 306, the hyperpolarized Xe129 is introduced into the lung. At step 308, shortly after introduction during a period of breath-hold, diffusion sensitized images are acquired by using a gradient-echo pulse sequence that incorporates a bipolar gradient waveform just after the excitation radio-frequency pulse for diffusion sensitization. At step 310, spatial maps of the ADC values are calculated from the diffusion-sensitized images using linear least squares fitting of the natural logarithm of the signal intensities versus b value on a pixel-by-pixel basis. At step 312, the ADC values in the area of interest are then evaluated by comparing them to normative ADC values.

Practice of various embodiments will be still more fully understood from the following examples, which are presented herein for illustration only and should not be construed as limiting the invention in any way.

### EXAMPLE No. 1

An aspect of an embodiment of the method for detecting or assessing changes in lung-tissue in a lung comprises: a) generating hyperpolarized Xe 129 gas; b) introducing the gas into the lung after the lung is positioned within an appropriate radio-frequency coil that is within a magnetic resonance imaging (MRI) apparatus; c) acquiring at least two magnetic resonance signals from Xe129 nuclei within the lung wherein the signals are “diffusion sensitized” such that the value of a property of the signals varies between the signals and reflects, among other possible effects, the degree to which diffusion has affected the signals; d) calculating apparent diffusion coefficient values from the diffusion-sensitized magnetic resonance signals; and e) evaluating apparent diffusion coefficient values.

Still referring to this exemplary method, generating the gas may be accomplished by optical pumping and spin exchange. Also, in addition to the hyperpolarized Xe129 at least one other gas may be introduced into the lung, wherein the other gas may have the purpose of modifying the apparent diffusion coefficient of the hyperpolarized Xe129. The gas's volume and nuclear polarization may be chosen based on at least one of the volume of the lung, and the desired spatial resolution, desired temporal resolution and desired signal-to-noise ratio of magnetic resonance signals to be generated. Also, introducing of the gas into the lung is by inhalation from a plastic bag, inhalation from a computer controlled gas mixing system, introduction by depressing a gas-filled syringe, or introduction by using a computer-controlled or manually-controlled ventilation device. Further, acquiring of the diffusion-sensitized magnetic resonance signals may occur during inhalation, during exhalation, during breath-holding, or some combination thereof. Calculating of the apparent diffusion coefficient values from the diffusion-sensitized magnetic resonance signals from Xe129 nuclei may include correcting the signals for the extraneous effect(s) of at least one of T1 decay, T2 decay, T2* decay and RF pulses. In an approach, at least two values of diffusion sensitization may be used. The method of diffusion sensitization may involve modulating the phase of the transverse magnetization along at least one spatial direction by using at least one magnetic field gradient pulse, wherein a bipolar magnetic field gradient pulse is used to modulate the phase of the transverse magnetization. Further, a time delay may be inserted between the positive and negative portions of the bipolar magnetic field gradient pulse. It should be appreciated that to achieve a higher degree of diffusion sensitization while maintaining a chosen fundamental time period of diffusion sensitization, the bipolar magnetic field gradient pulse is applied at least twice prior to acquiring a given diffusion-sensitized magnetic resonance signal. The bipolar gradients of opposite senses may be applied back-to-back to achieve compensation for bulk motion. The method of diffusion sensitization may involve modulating the amplitude of the longitudinal magnetization along at least one spatial direction by using at least two radio-frequency pulses interspersed with at least one magnetic field gradient pulse. The effect of diffusion on the modulated longitudinal magnetization may be monitored by acquiring at least two magnetic resonance images that are separated by appropriately chosen time delays. The diffusion-sensitized magnetic resonance signals may reflect the signal from all Xe129 nuclei within the lung. The diffusion-sensitized magnetic resonance signals may reflect the signal from Xe129 nuclei within one or more selected sub-volumes within the whole of the lung, wherein each the sub-volume may correspond to a planar slice of lung tissue, a column of lung tissue, or some arbitrarily-shaped volume of lung tissue. The property of the diffusion-sensitized magnetic resonance signals that reflects the effect of diffusion is the amplitude of the signals. At least one magnetic field gradient pulse may be applied for at least one of before and during the acquiring of the diffusion-sensitized magnetic resonance signals in any manner consistent with imaging pulse sequences known in the art to permit a diffusion-sensitized magnetic resonance image, resolved in one, two or three spatial dimensions, to be calculated. The diffusion-sensitized magnetic resonance images may be acquired corresponding to one or more spatial locations. The calculating of the apparent diffusion coefficient values yields spatially resolved maps of the values. The acquiring of the diffusion-sensitized magnetic resonance images is performed by using a gradient-echo pulse sequence. The gradient-echo pulse sequence incorporates a bipolar gradient waveform just after the excitation radio-frequency pulse for diffusion sensitization. The calculating of the apparent diffusion coefficient values may be performed from the signals corresponding to the different diffusion sensitizations by using linear least squares fitting of the natural logarithm of the signal intensities versus the degree of diffusion sensitization. The lung may be the lung of an animal or of a human, wherein the lung may be in vivo or excised.

### EXAMPLE No. 2

An aspect of an embodiment of the MRI apparatus for detecting or assessing changes in lung-tissue using hyperpolarized Xe129 gas comprises: a) a radio frequency coil, wherein the gas is introduced into the lung and the lung is positioned within the radio-frequency coil; b) an MR images acquisition means, wherein the MR images acquisition means acquiring at least two magnetic resonance signals from Xe129 nuclei within the lung wherein the signals are “diffusion sensitized” such that the value of a property of the signals varies between the signals and reflects, among other possible effects, the degree to which diffusion has affected the signals; c) calculating means, wherein the calculating means for calculating apparent diffusion coefficient values from the diffusion-sensitized resonance signals; and d) an evaluating means, wherein the evaluating means for evaluating coefficient values.

Still referring to the exemplary apparatus, the apparatus may further comprise a gas generating means for providing the hyperpolarized Xe129 gas, wherein the generating means may hyperpolarize the gas by optical pumping and spin exchange. In addition to the hyperpolarized Xe129 at least one other gas may be introduced into the lung, wherein the other gas may have the purpose of modifying the apparent diffusion coefficient of the hyperpolarized Xe129. Also, the gas's volume and nuclear polarization may be chosen based on at least one of the volume of the lung, and the desired spatial resolution, desired temporal resolution and desired signal-to-noise ratio of magnetic resonance signals to be generated. Further, the gas introduced into the lung may be by inhalation from a plastic bag, inhalation from a computer controlled gas mixing system, introduction by depressing a gas-filled syringe, or introduction by using a computer-controlled or manually-controlled ventilation device. The acquisition of the diffusion-sensitized magnetic resonance signals may occur during inhalation, during exhalation, during breath-holding, or some combination thereof. Further, the calculation of the apparent diffusion coefficient values from the diffusion-sensitized magnetic resonance signals from Xe129 nuclei may include correcting the signals for the extraneous effect(s) of at least one of T1 decay, T2 decay, T2* decay and RF pulses. Also, at least two values of diffusion sensitization may be used. The diffusion-sensitized signals may involve modulating the phase of the transverse magnetization along at least one spatial direction by using at least one magnetic field gradient pulse. A bipolar magnetic field gradient pulse may be used to modulate the phase of the transverse magnetization. A time delay may be inserted between the positive and negative portions of the bipolar magnetic field gradient pulse. In order to achieve a higher degree of diffusion sensitization while maintaining a chosen fundamental time period of diffusion sensitization, the bipolar magnetic field gradient pulse may be applied at least twice prior to acquiring a given diffusion-sensitized magnetic resonance signal. The bipolar gradients of opposite senses may be applied back-to-back to achieve compensation for bulk motion. In addition, the diffusion-sensitized signals involves modulating the amplitude of the longitudinal magnetization along at least one spatial direction by using at least two radio-frequency pulses interspersed with at least one magnetic field gradient pulse. It should be appreciated that the effect of diffusion on the modulated longitudinal magnetization may be monitored by acquiring at least two magnetic resonance images that are separated by appropriately chosen time delays. The diffusion-sensitized magnetic resonance signals may reflect the signal from all Xe129 nuclei within the lung. The diffusion-sensitized magnetic resonance signals may reflect the signal from Xe129 nuclei within one or more selected sub-volumes within the whole of the lung, wherein each the sub-volume may correspond to a planar slice of lung tissue, a column of lung tissue, or some arbitrarily-shaped volume of lung tissue. The property of the diffusion-sensitized magnetic resonance signals that reflects the effect of diffusion may be the amplitude of the signals. Further, at least one magnetic field gradient pulse may be applied for at least one of before and during the acquisition of the diffusion-sensitized magnetic resonance signals in any manner consistent with imaging pulse sequences known in the art to permit a diffusion-sensitized magnetic resonance image, resolved in one, two or three spatial dimensions, to be calculated. Still yet, the diffusion-sensitized magnetic resonance images may be acquired corresponding to one or more spatial locations. It should be appreciated that the calculation of the apparent diffusion coefficient values may yield spatially resolved maps of the values. The acquisition of the diffusion-sensitized magnetic resonance images may be performed by using a gradient-echo pulse sequence. The gradient-echo pulse sequence may incorporate a bipolar gradient waveform just after the excitation radio-frequency pulse for diffusion sensitization. The calculation of the apparent diffusion coefficient values may be performed from the signals corresponding to the different diffusion sensitizations by using linear least squares fitting of the natural logarithm of the signal intensities versus the degree of diffusion sensitization. Also, it should be appreciated that the lung may be the lung of an animal or of a human, wherein the lung may be in vivo or excised.

It should be understood that while the method described was presented with a certain ordering of the steps, it is not our intent to in any way limit the present invention to a specific step order. It should be appreciated that the various steps can be performed in different orders, for example, the step of positioning the lung in the MR system may occur prior to, or simultaneously with, the generation of the hyperpolarized Xe129. Further, we have described herein the novel features of the present invention, and it should be understood that we have not included details well known by those of skill in the art, such as the design and operation of a MR imaging system.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of the appended claims. For example, regardless of the content of any portion (e.g., title, section, abstract, drawing figure, etc.) of this application, unless clearly specified to the contrary, there is no requirement for any particular described or illustrated activity or element, any particular sequence of such activities, any particular size, speed, dimension or frequency, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Accordingly, the descriptions and drawings are to be regarded as illustrative in nature, and not as restrictive.

We claim:

1. A method for detecting or assessing changes in lung-tissue in a lung, said method comprising:
generating hyperpolarized Xe129 gas;
introducing said gas into the lung after the lung is positioned within an appropriate radio-frequency coil that is within a magnetic resonance imaging (MRI) apparatus;
acquiring at least two magnetic resonance signals from Xe129 nuclei within the lung wherein said signals are “diffusion sensitized” such that the value of a property of said signals varies between the signals and reflects, among other possible effects, the degree to which diffusion has affected said signals;
calculating apparent diffusion coefficient values from said diffusion-sensitized magnetic resonance signals; and
evaluating apparent diffusion coefficient values.

2. The method of claim 1, wherein said generating said gas is by optical pumping and spin exchange.

3. The method of claim 1, wherein in addition to said hyperpolarized Xe129 at least one other gas is introduced into said lung.

4. The method of claim 3, wherein said at least one other gas have the purpose of modifying the apparent diffusion coefficient of said hyperpolarized Xe129.

5. The method of claim 1, wherein said gas's volume and nuclear polarization are chosen based on at least one of the volume of the lung, and the desired spatial resolution, desired temporal resolution and desired signal-to-noise ratio of magnetic resonance signals to be generated.

6. The method of claim 1, wherein said introducing of said gas into the lung is by inhalation from a plastic bag, inhalation from a computer controlled gas mixing system, introduction by depressing a gas-filled syringe, or introduction by using a computer-controlled or manually-controlled ventilation device.

7. The method of claim 1, wherein said acquiring of said diffusion-sensitized magnetic resonance signals occurs during inhalation, during exhalation, during breath-holding, or some combination thereof.

8. The method of claim 1, wherein said calculating of said apparent diffusion coefficient values from said diffusion-sensitized magnetic resonance signals from Xe129 nuclei includes correcting said signals for the extraneous effect(s) of at least one of T1 decay, T2 decay, T2* decay and RF pulses.

9. The method of claim 1, wherein at least two values of diffusion sensitization are used.

10. The method of claim 1, wherein the method of diffusion sensitization involves modulating the phase of the transverse magnetization along at least one spatial direction by using at least one magnetic field gradient pulse.

11. The method of claim 10, wherein a bipolar magnetic field gradient pulse is used to modulate the phase of the transverse magnetization.

12. The method of claim 11, wherein a time delay is inserted between the positive and negative portions of the bipolar magnetic field gradient pulse.

13. The method of claim 11, wherein, to achieve a higher degree of diffusion sensitization while maintaining a chosen fundamental time period of diffusion sensitization, the bipolar magnetic field gradient pulse is applied at least twice prior to acquiring a given diffusion-sensitized magnetic resonance signal.

14. The method of claim 11, wherein bipolar gradients of opposite senses are applied back-to-back to achieve compensation for bulk motion.

15. The method of claim 1, wherein the method of diffusion sensitization involves modulating the amplitude of the longitudinal magnetization along at least one spatial direction by using at least two radio-frequency pulses interspersed with at least one magnetic field gradient pulse.

16. The method of claim 15, wherein the effect of diffusion on the modulated longitudinal magnetization is monitored by acquiring at least two magnetic resonance images that are separated by appropriately chosen time delays.

17. The method of claim 1, wherein said diffusion-sensitized magnetic resonance signals reflect the signal from all Xe129 nuclei within the lung.

18. The method of claim 1, wherein said diffusion-sensitized magnetic resonance signals reflect the signal from Xe129 nuclei within one or more selected sub-volumes within the whole of the lung, wherein each said sub-volume may correspond to a planar slice of lung tissue, a column of lung tissue, or some arbitrarily-shaped volume of lung tissue.

19. The method of claim 1, wherein said property of said diffusion-sensitized magnetic resonance signals that reflects the effect of diffusion is the amplitude of the signals.

20. The method of claim 1, wherein at least one magnetic field gradient pulse is applied for at least one of before and during the acquiring of said diffusion-sensitized magnetic resonance signals in any manner consistent with imaging pulse sequences known in the art to permit a diffusion-sensitized magnetic resonance image, resolved in one, two or three spatial dimensions, to be calculated.

21. The method of claim 20, wherein diffusion-sensitized magnetic resonance images are acquired corresponding to one or more spatial locations.

22. The method of claim 20, wherein said calculating of said apparent diffusion coefficient values yields spatially resolved maps of said values.

23. The method of claim 22, wherein said acquiring of said diffusion-sensitized magnetic resonance images is performed by using a gradient-echo pulse sequence.

24. The method of claim 23, wherein said gradient-echo pulse sequence incorporates a bipolar gradient waveform just after the excitation radio-frequency pulse for diffusion sensitization.

25. The method of claim 1, wherein said calculating of said apparent diffusion coefficient values is performed from the signals corresponding to the different diffusion sensitizations by using linear least squares fitting of the natural logarithm of the signal intensities versus the degree of diffusion sensitization.

26. The method of claim 1, wherein the lung is the lung of an animal or of a human.

27. The method of claim 26, wherein the lung may be in vivo or excised.

28. An MRI apparatus for detecting or assessing changes in lung-tissue structure of a lung using hyperpolarized Xe129 gas, said apparatus comprising:
a radio frequency coil, wherein the gas is introduced into the lung and the lung is positioned within said radio-frequency coil;
an MR images acquisition means, said MR images acquisition means acquiring at least two magnetic resonance signals from Xe129 nuclei within the lung wherein said signals are “diffusion sensitized” such that the value of a property of said signals varies between the signals and reflects, among other possible effects, the degree to which diffusion has affected said signals;
calculating means, said calculating means for calculating apparent diffusion coefficient values from said diffusion-sensitized resonance signals; and
an evaluating means, said evaluating means for evaluating coefficient values.

29. The apparatus of claim 28, further comprising a gas generating means for providing the hyperpolarized Xe129 gas.

30. The apparatus of claim 29, wherein said generating means hyperpolarizes said gas by optical pumping and spin exchange.

31. The apparatus of claim 28, wherein in addition to said hyperpolarized Xe129 at least one other gas is introduced into said lung.

32. The apparatus of claim 31, wherein said at least one other gas have the purpose of modifying the apparent diffusion coefficient of said hyperpolarized Xe129.

33. The apparatus of claim 28, wherein said gas's volume and nuclear polarization are chosen based on at least one of the volume of the lung, and the desired spatial resolution, desired temporal resolution and desired signal-to-noise ratio of magnetic resonance signals to be generated.

34. The apparatus of claim 28, wherein said gas introduced into the lung is by inhalation from a plastic bag, inhalation from a computer controlled gas mixing system, introduction by depressing a gas-filled syringe, or introduction by using a computer-controlled or manually-controlled ventilation device.

35. The apparatus of claim 28, wherein said acquisition of said diffusion-sensitized magnetic resonance signals occurs during inhalation, during exhalation, during breath-holding, or some combination thereof.

36. The apparatus of claim 28, wherein said calculation of said apparent diffusion coefficient values from said diffusion-sensitized magnetic resonance signals from Xe129 nuclei includes correcting said signals for the extraneous effect(s) of at least one of T1 decay, T2 decay, T2* decay arid RF pulses.

37. The apparatus of claim 28, wherein at least two values of diffusion sensitization are used.

38. The apparatus of claim 28, wherein said diffusion-sensitized signals involves modulating the phase of the transverse magnetization along at least one spatial direction by using at least one magnetic field gradient pulse.

39. The apparatus of claim 38, wherein a bipolar magnetic field gradient pulse is used to modulate the phase of the transverse magnetization.

40. The apparatus of claim 39, wherein a time delay is inserted between the positive and negative portions of the bipolar magnetic field gradient pulse.

41. The apparatus of claim 39, wherein, to achieve a higher degree of diffusion sensitization while maintaining a chosen fundamental time period of diffusion sensitization, the bipolar magnetic field gradient pulse is applied at least twice prior to acquiring a given diffusion-sensitized magnetic resonance signal.

42. The apparatus of claim 39, wherein bipolar gradients of opposite senses are applied back-to-back to achieve compensation for bulk motion.

43. The apparatus of claim 28, wherein the diffusion-sensitized signals involves modulating the amplitude of the longitudinal magnetization along at least one spatial direction by using at least two radio-frequency pulses interspersed with at least one magnetic field gradient pulse.

44. The apparatus of claim 43, wherein the effect of diffusion on the modulated longitudinal magnetization is monitored by acquiring at least two magnetic resonance images that are separated by appropriately chosen time delays.

45. The apparatus of claim 28, wherein said diffusion-sensitized magnetic resonance signals reflect the signal from all Xe129 nuclei within the lung.

46. The apparatus of claim 28, wherein said diffusion-sensitized magnetic resonance signals reflect the signal from Xe129 nuclei within one or more selected sub-volumes within the whole of the lung, wherein each said sub-volume may correspond to a planar slice of lung tissue, a column of lung tissue, or some arbitrarily-shaped volume of lung tissue.

47. The apparatus of claim 28, wherein said property of said diffusion-sensitized magnetic resonance signals that reflects the effect of diffusion is the amplitude of the signals.

48. The apparatus of claim 28, wherein at least one magnetic field gradient pulse is applied for at least one of before and during the acquiring of said diffusion-sensitized magnetic resonance signals in any manner consistent with imaging pulse sequences known in the art to permit a diffusion-sensitized magnetic resonance image, resolved in one, two or three spatial dimensions, to be calculated.

49. The apparatus of claim 48, wherein diffusion-sensitized magnetic resonance images are acquired corresponding to one or more spatial locations.

50. The apparatus of claim 48, wherein said calculating of said apparent diffusion coefficient values yields spatially resolved maps of said values.

51. The apparatus of claim 50, wherein said acquiring of said diffusion-sensitized magnetic resonance images is performed by using a gradient-echo pulse sequence.

52. The apparatus of claim 51, wherein said gradient-echo pulse sequence incorporates a bipolar gradient waveform just after the excitation radio-frequency pulse for diffusion sensitization.

53. The apparatus of claim 28, wherein said calculation of said apparent diffusion coefficient values is performed from the signals corresponding to the different diffusion sensitizations by using linear least squares fitting of the natural logarithm of the signal intensities versus the degree of diffusion sensitization.

54. The apparatus of claim 28, wherein the lung is the lung of an animal or of a human.

55. The method of claim 54, wherein the lung may be in vivo or excised.
